(19) United States
(12) Patent Application Publication
(10) Pub. No.: US 2026/0022320 A1
(43) Pub. Date: Jan. 22, 2026

(54) CELL RETENTION APPARATUS WITH CONVECTIVE FLOW

(71) Applicant: Board of Trustees of Michigan State University, East Lansing, MI (US)

(72) Inventors: Brian P. JOHNSON, Lansing, MI (US); Jacob I. REYNOLDS, Lansing, MI (US)

(73) Assignee: Board of Trustees of Michigan State University, East Lansing, MI (US)

(21) Appl. No.: 19/275,236

(22) Filed: Jul. 21, 2025

Related U.S. Application Data
(60) Provisional application No. 63/674,026, filed on Jul. 22, 2024.

(51) Int. Cl. C12M 1/34; C12M 1/32; C12M 1/36

(52) U.S. Cl. CPC C12M 41/12; C12M 23/12; C12M 41/48

(57) ABSTRACT

A cell retention apparatus with convective flow is provided. In another aspect, a microplate-based array of microfluidic wells has a heater or cooler which generates controllable temperature gradients within and/or between the wells, such as in an injection molded and/or disposable cell culture well plate. These gradients create convection currents within the well that generate flow or within connecting microchannels between wells. A further aspect induces controlled thermal convection currents within wells of a microtiter plate to drive fluid movement.

### CROSS-REFERENCE TO RELATED APPLICATION

This application claims priority to U.S. patent application Ser. No. 63/674,026, filed on Jul. 22, 2024, which is incorporated by reference herein.

### GOVERNMENT SUPPORT

This invention was made with government support under R00-ES028744 awarded by the National Institute of Health and P42ES004911 awarded by the National Institute of Environmental Health Sciences. The government has certain rights in the invention.

### BACKGROUND

The present application generally pertains to heating and/or cooling of a cell and liquid retention apparatus, and more particularly to a cell culture plate apparatus with convective flow.

Cell culture plates having wells therein are well known. This is shown in U.S. Pat. No. 11,745,183 entitled “Microtiter Plate and Uses Thereof,” which issued to Jimenez, Johnson and Beebe on Sep. 5, 2023. This patent is incorporated by reference herein.

Cell cultures lack many physiologically important features from their normal environment in vivo. More physiologically relevant new approach methods are needed to improve predictability for drug and chemical safety testing. Fluid flow influences cellular behavior in vivo and introducing flow to a micro-physiological model can be accomplished in several ways including by adding pumps, incorporating hydraulic heads or rocking the devices, but all of these traditional attempts add complexity, decrease throughput or otherwise interfere with analyses.

A known system is disclosed in U.S. Pat. No. 6,663,757 entitled “Method and Device for the Convective Movement of Liquids in Microsystems,” which issued to Fuhr, et al., employes a DNA chip using laser induced thermal gradients to cause convective blending of fluids. Another conventional device is discussed in U.S. Patent Publication No. 2006/0057029 entitled “Analytical Biochemistry System with Robotically Carried Bioarray,” which published to Coassin, et al., generates a thermal gradient via electrical current, electrical resistance, junction heaters, IR radiation or RF to cause convective flow in liquid samples. This patent and patent publication are incorporated by reference herein. However, these conventional configurations are overly complex and also interfere with easy sample analysis.

### SUMMARY

In accordance with the present invention, a cell retention apparatus, such as a biological cell culture plate apparatus with convective flow, is provided. A method of causing convective flow in a cell culture plate is also provided. In another aspect, a microplate-based array of microfluidic wells has a heater or cooler which generates controllable temperature gradients within and/or between the wells, such as in a low-cost, injection molded and/or disposable cell culture well plate. These gradients create convection currents within the well that generate flow or within connecting microchannels between wells. A further aspect induces controlled thermal convection currents within wells of a microtiter plate to drive fluid movement.

Another aspect creates different temperate gradients for one well, multiple wells, selected well(s) and/or each of the wells, from an array of many rows and columns of wells on the plate. Still another aspect actively controls the temperature gradient and/or convective flow in a predetermined manner, in one well, multiple wells, selected well(s) and/or each of the wells, from an array of many rows and columns of wells on the plate. In yet an additional aspect, the present cell culture plate apparatus and method include a thermal convection flow inducing heater/cooler, such as: (a) a well or depressed plate cavity with an evaporate liquid or desiccant therein, adjacent a sample well; (b) a heated pin or rib in or adjacent to a sample well; (c) a thermal channel flowing a cooling and/or heating fluid between sample wells; (d) heated wires extending between sample wells; and/or (e) a stationary plate holder or support including elongated heating elements spanning between upstanding walls upon which a cell culture plate is removably placed.

The present cell retention apparatus and method are advantageous over conventional systems. For example, the present apparatus and method are lower cost, less complex and do not interfere with easy sample analysis in the wells, as compared to traditional devices. In certain embodiments, the fluid flow is generated without the need for tubes, pumps or other coupled hardware, to add physiological relevance, such as flow in an in vitro blood vessel. Moreover, the present apparatus and method are applicable to microphysiological model creation, improved formation of spheroid models, the addition of physiological flow rates in microvessels or microchannels, and reduced thermal edge effects due to traditional uneven plate heating.

In some configurations, the present apparatus and method improved consistency since cells are maintained in suspension which facilitates even seeding density across the plate. In some configuration, the present apparatus and method beneficially provide unidirectional cooling or heating which may improve cell viability and/or facilitate chemical mixing. Furthermore, the present apparatus and method achieve contactless mixing of the liquid composition in the well(s). In other configurations, the present apparatus and method advantageously facilitate mixing of reagents without physical manipulation, which prevents sloshing or spilling. In an optional arrangement, the vessel or well remains stationery and is uncapped, thereby facilitating robotic sampling, reaction monitoring and sensing. Additional benefits and features of the present apparatus and method will become apparent from the following description and appended figures.

### BRIEF DESCRIPTION OF THE DRAWINGS

FIG. 1 is a diagrammatic side view showing a first plate embodiment of the present cell retention apparatus;

FIG. 2 is a diagrammatic side view showing a second plate embodiment of the present cell retention apparatus;

FIGS. 3 and 4 are cross-sections, taken along lines 3-3 and 4-4, respectively, from FIG. 1, applicable to the first, second and third plate embodiments of the present cell retention apparatus;

FIG. 5 is a diagrammatic side view showing a third plate embodiment of the present cell retention apparatus;

FIG. 6 is a diagrammatic side view showing a fourth plate embodiment of the present cell retention apparatus;

FIG. 7 shows exploded and assembled perspective views of a serpentine fourth plate embodiment of the present cell retention apparatus;

FIG. 8 is a diagrammatic top view showing a pin heating embodiment of the present cell retention apparatus;

FIG. 9 is a diagrammatic side view showing the pin heating embodiment of the present cell retention apparatus;

FIG. 10 is a diagrammatic side view showing a pin embodiment of the present cell retention apparatus;

FIG. 11 is a diagrammatic side view showing a pin embodiment of the present cell retention apparatus;

FIG. 12 is a diagrammatic side view showing a pin embodiment of the present cell retention apparatus;

FIG. 13 is a diagrammatic side view showing a serpentine embodiment of the present cell retention apparatus;

FIG. 14 is a diagrammatic side view showing the serpentine embodiment of the present cell retention apparatus;

FIG. 15 is a diagrammatic side view showing functionality of the first plate embodiment of the present cell retention apparatus;

FIG. 16 is a diagrammatic side view showing functionality of the first plate embodiment of the present cell retention apparatus;

FIG. 17 is a diagrammatic side view showing functionality of the first plate embodiment of the present cell retention apparatus;

FIGS. 18 and 19 are diagrammatic views showing forces on a cell employed in the first plate embodiment of the present cell retention apparatus;

FIG. 20 is a diagrammatic top view showing functionality of the first embodiment of the present cell retention apparatus;

FIG. 21 is a diagrammatic perspective view showing the functionality of the first embodiment of the present cell retention apparatus;

FIGS. 22-24 are graphs showing expected results of the first embodiment of the present cell retention apparatus;

FIG. 25 is a diagrammatic perspective view showing a fifth plate embodiment of the present cell retention apparatus;

FIG. 26 is a diagrammatic perspective view showing a sixth plate embodiment of the present cell retention apparatus;

FIG. 27 is a diagrammatic perspective view showing a seventh plate embodiment of the present cell retention apparatus;

FIGS. 28 and 29 are diagrammatic views showing a seventh plate embodiment of the present cell retention apparatus;

FIG. 30 is a diagrammatic view showing a serpentine embodiment of the present cell retention apparatus;

FIGS. 31-38 are diagrammatic views showing an eighth plate embodiment of the present cell retention apparatus;

FIG. 39-42 are fluorescent microscopic images showing the present cell retention apparatus;

FIG. 43 is a diagrammatic side view showing a ninth embodiment of the present cell retention apparatus;

FIG. 44 is a cross-sectional view, taken along line 44-44 from FIG. 43, showing the ninth embodiment of the present cell retention apparatus; and

FIG. 45 is an exploded perspective view showing a tenth embodiment of the present cell retention apparatus.

### DETAILED DESCRIPTION

The present cell retention apparatus and method of using such induces flow within a microplate-based array of microfluidic devices. Preferably, the cell retention apparatus is a biological cell culture plate apparatus. For example, but not limitation, the present apparatus and method control thermal convection currents within wells of a microtiter plate to drive fluid movement. Using a variety of device designs, convection currents can be induced through local heating of devices outside a microplate or by adding features to a microplate including integrated heating/cooling channels, adding exothermic and/or endothermic processes in adjacent wells.

Directed flow is used to improve physiological relevance of cell culture models. Furthermore, the present apparatus is biocompatible, simple without attached tubes, pumps or other added hardware, and is throughput compatible by being well plate based. The thermal convection currents drive fluid flow within wells and/or between wells, such as through microchannels. The thermal convention currents optionally improve spheroid formation in the wells.

The present apparatus and method are ideally suited for use in biological processes. For example, analysis of blood clotting and coagulation with different static and/or flow conditions. For example, analysis of removal of cellular waste and/or CO₂. For example, analysis of increasing O₂ in the sample. For example, analysis of creating shear stress via endothelial dynamics and/or epithelial dynamics. For example, analysis of immune invasion such as cancer cell extravasation and invasion. For example, analysis of lymphatic fluid dynamics. Also for example, analysis of milk duct collection.

In an optional configuration, the present apparatus and method provide cell culture support devices that utilize a thermal gradient to maintain cells in suspension to facilitate homogenous sampling and/or mixing. A thermal gradient is induced in the liquid from outside the vessel causing convection currents to form in the liquid media. Engineering the location of these convection currents can drive flow within the media as changing densities cause the warmer liquid to rise and the cooler liquid to sink.

In another optional configuration, the present apparatus and method may apply a thermal gradient to a cell/reagent reservoir to maintain cells in suspension for even sampling and seeding into microplates. This technology addresses an established problem of cells quickly settling to the bottom leading to uneven numbers of cells seeded across the plate.

The present apparatus and method create convection currents that are generated by small temperature gradients in a liquid to drive fluid flow in a simple, contactless and biocompatible manner. This technology is hereby applied to cell culture microplates where mixing is induced by a tuned circular flow within a well or drive flow through engineered channels connecting multiple wells. Flow is a desirable physiological parameter that is often lacking in traditional microphysiological systems where it serves to bring oxygen and remove waste. The present convection flow is in contrast to conventional attempts at introducing flow which add too much complexity, thus reducing throughput (such as by integration with pumps, tubes, etc.), or interfere with real-time monitoring (such as by using shaking or rocking motion).

Certain aspects of the present apparatus and method provide contactless mixing of cell culture media/reactants to maintain homogeneity. Other aspects provide contactless mixing of chemical reactants to speed reactions. Yet other aspects generate tunable flow without the need for tubes, pumps, or other added hardware. When the present apparatus is applied in microfluidic, organ-on-a-chip and other microphysiological devices, benefits of the present convection flow include removal of waste and increased oxygenation, model immune invasion or cancer cell extravasation/invasion, addition of shear stress (epithelial and endothelial dynamics), lymphatic dynamics, and the like. In further aspects of the present apparatus and method, directional and tunable flow rates achieve different physiologies/use cases. Other optional features of the present apparatus and method include suspending a cell/spheroid culture, maintaining cell suspension in a sampling reservoir, reducing edge effects where the process occurs naturally but is uncontrolled, and/or maintaining cell suspension for sampling by a flow cytometer.

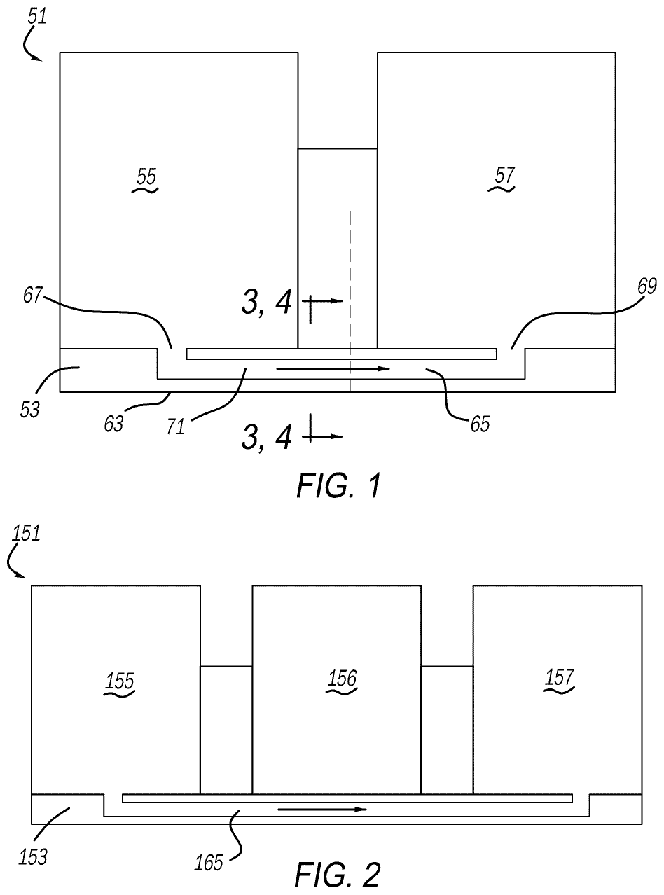

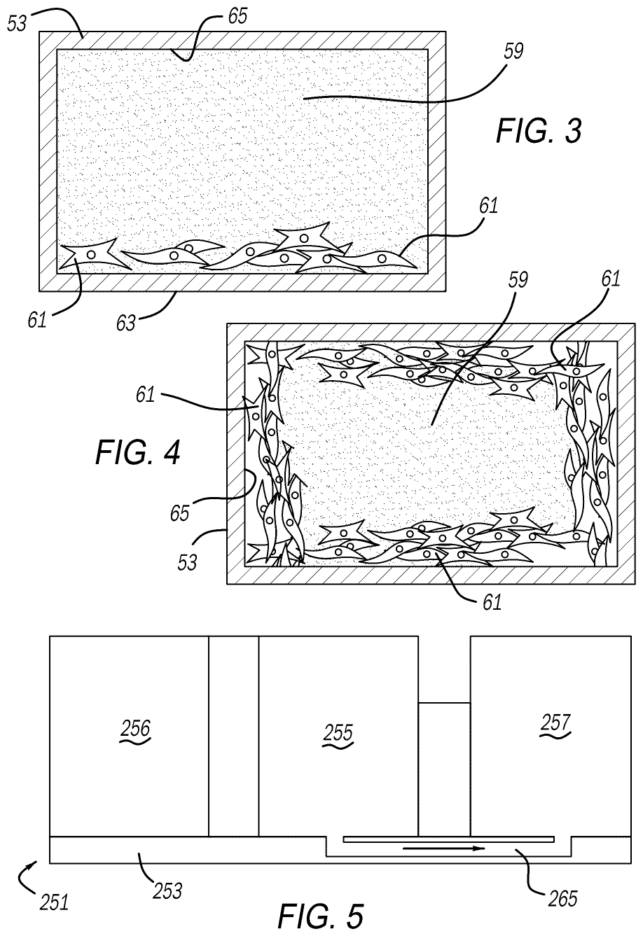

A first plate embodiment of the present apparatus 51 employs evaporative cooling. This can be observed in FIGS. 1 and 3. In this arrangement, a cell culture plate 53 (also known as a tray) includes wells 55 and 57 containing a coolant liquid 59, such as ethanol, with a greater rate of evaporation than that of the specimen fluid which includes biological cells 61. Each well is a concave or recessed cavity open at a top surface of plate 53 and there is a generally flat bottom surface 63 on the opposite face of the plate. There are multiple such wells 55 and 57 aligned in columns and rows within the plate; for example, there preferably at least 100 wells in the plate and more preferably 384 wells.

Adjacent wells with cooler (e.g., well 55) and warmer (e.g., well 57) specimen fluids are connected by a microchannel 65. Microchannel 65 is preferably integrally formed as a single piece within the bottom wall of plate 53, having inlet and outlet ports 67 and 69, respectively, openly accessible to a bottom of each well 55 and 57, respectively. A laterally elongated passageway 71 extends between the ports and the passageway is at least ten time longer than wider in the present nonlimiting example. The microchannel may be formed by creating a void layer between additively manufactured, such as by three-dimensionally printed polymeric or alternately, metallic, layers below and partially above the void layer.

The microplate-based array of microfluidic wells has a heater which generates controllable temperature gradients within and/or between the wells, such as in a standard, low-cost, injection molded and/or disposable cell culture well plate. Various heater configurations will be discussed in greater detail hereinafter. If plate 53 is injection molded, then the bottom wall of the plate may be made in two assembled pieces to allow a molding die lower access to form the upper walls, side walls and ports for the microchannels 65 integral with an upper segment of the plate, and with a bottom wall segment thereafter attached to form the bottom wall of the microchannels. These gradients create convection currents within the well that generate flow or within connecting microchannels between wells.

FIG. 2 shows a second plate embodiment of the present apparatus 151. This construction includes a cell culture plate 153 with wells 155, 156 and 157 formed therein for holding a cell and liquid solution. Well 155 is of a cooler temperature than non-adjacent well 157, with a nonaffiliated well 156 laterally positioned therebetween. A microchannel 165 is located in the bottom wall of plate 153, below the wells, and connects between cool well 155 and warm well 157 to cause convective flow of the solution between the connected wells and underneath intermediate well 156. Well 156 may be optionally connected with an offset angled (such as laterally perpendicular) microchannel to a fourth well.

A third plate embodiment of the present apparatus 251 can be observed in FIG. 5. This embodiment includes a cell culture plate 253 with wells 255, 256 and 257 formed therein for holding a cell and liquid solution. Wells 255 and 256 are of a cooler temperature than well 257. Well 256 is nonaffiliated and laterally outboard of wells 255 and 257. Moreover, well 256 preferably contains a liquid with a higher evaporation rate than well 255 to aid in cooling of well 255. A microchannel 265 is located in the bottom wall of plate 253, below wells 255 and 257, and connects between cool well 255 and warm well 257 to cause convective flow of the solution between the connected wells. Well 256 may optionally be connected with an offset angled (such as laterally perpendicular) microchannel to a fourth well.

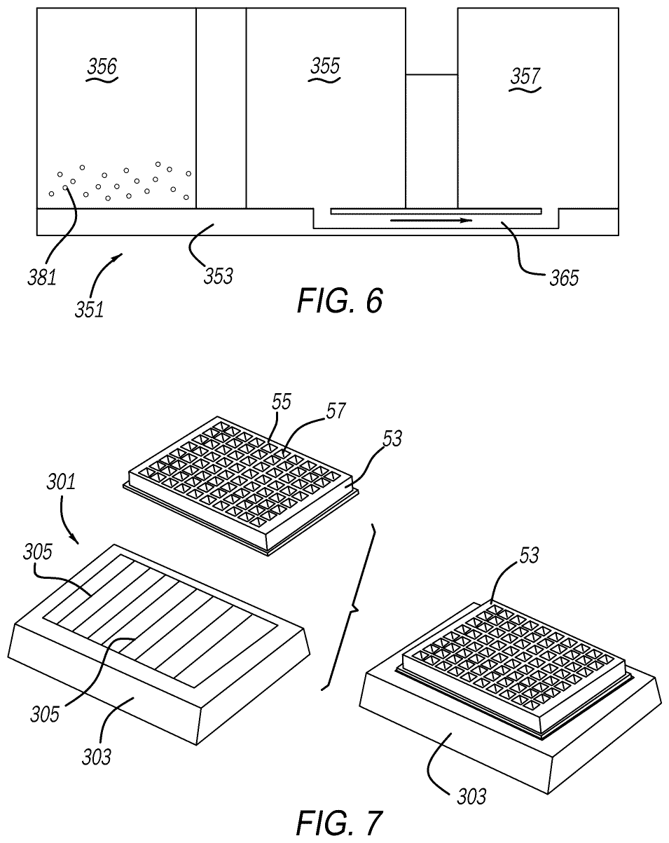

A fourth plate embodiment of the present apparatus 351 is shown in FIG. 6. Cool wells 355 and 356, and a warm well 357 are recessed in a cell culture plate 353. In this evaporative cooling arrangement, a desiccant material 381 is in well 356 adjacent to cooler and warmer specimen fluid wells, 355 and 357 respectively, that are connected by a microchannel 365. The desiccant serves as temperature control elements. The evaporative cooling embodiment cools (or alternate heats) an exothermal or endothermal reaction.

FIG. 7 illustrates an electronic temperature controller plate embodiment of the present apparatus. This embodiment of the present apparatus includes a heated plate base. Electricity is supplied from a power source through one or more heating wires, preferably parallel pairs of wires, between each row and/or column of wells. In an exemplary configuration, the wires are attached to a stationary base with wires spanning between upstanding walls, across the base. The plate is then removably placed on top of the base such that the exposed resistive wires create heat which is upwardly transmitted to the adjacent wells.

This approach is ideally suited for varying the heat applied to the middle wells on the plate versus the heat applied to the wells adjacent to the periphery of the plate. The heat differences may be caused by using outboard wires and inboard wires with different heating characteristics, such as resistance materials or sizes, or by changing the electrical current supplied to or deenergizing certain of the wires. Alternately, the wires can be molded within the plate.

A variation of the electronic temperature controller plate 301 includes a metallic plate holder or base 303 with multiple generally parallel heating elements or conduits, such as wires 305, spanning back and forth between opposite side thereof. Alternately, heating elements 305 may be connected tubes within which heating (or alternately cooling) fluid flows; the fluid being driven by a pump from a coupled fluid reservoir to manifolds, and externally heated (or cooled) in the reservoir by a heater or chiller. In use, plate 53 is directly placed on top of holder 303 so the heating elements are exposed to a bottom surface of the plate so the heat rises to the bottom of wells 55 and 57 (which may be interconnected or isolated). Cell culture plate 53 is preferably metallic in this configuration.

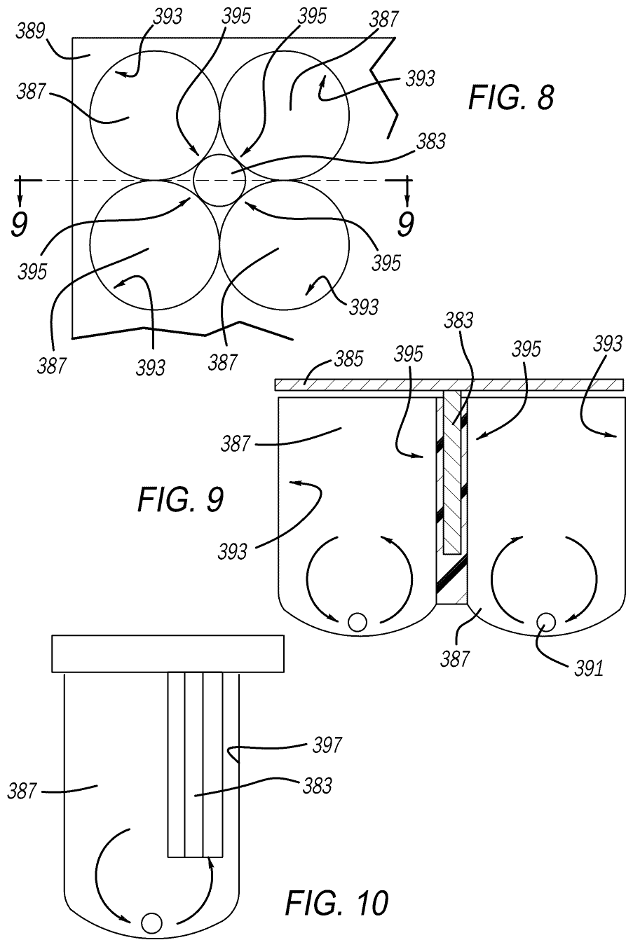

Reference should now be made to FIGS. 8 and 9. A heating pin embodiment of the present apparatus includes one or more heated pins 383 projecting from an electronic heater 385, into each well 387 or projecting into an upstanding wall of plate 389 between pairs of adjacent wells. The electronic heater includes electric or fluid heater elements, and have the multiple pins extending therefrom. Pins 383 are longitudinally elongated and are parallel to each other. The pins serve as temperature control elements.

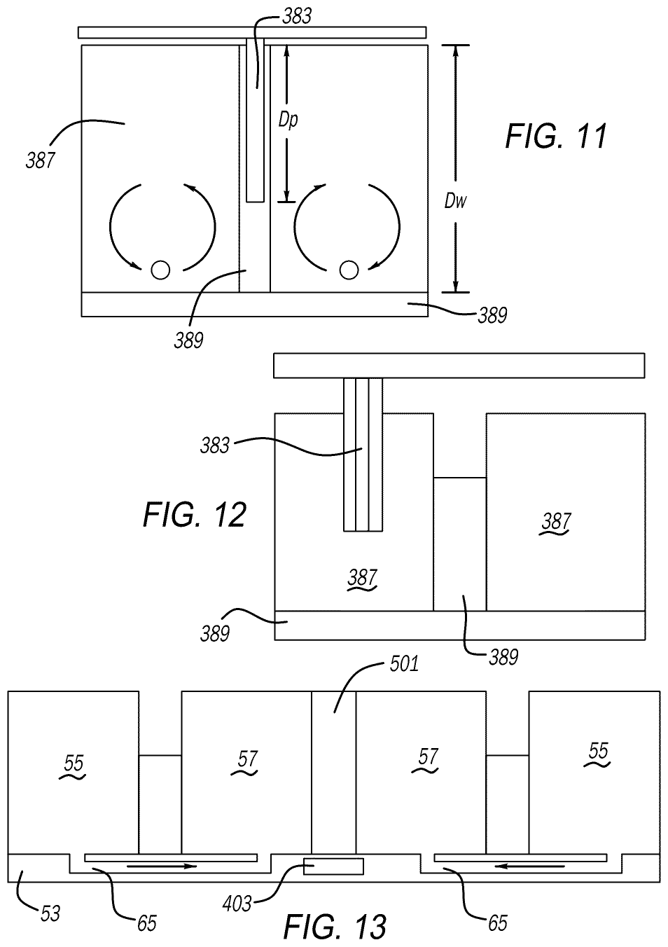

Furthermore, as can be observed in FIG. 11, a longitudinal depth D_{P} of the pins extend a majority of the longitudinal depth D_{w} of the associated well, but less than the entire depth, and the width of each pin is preferably less than a lateral wall thickness between adjacent of the wells. The pins cause convection within each well so that cells 391 therein move in a swirling motion between cooler and warmer zones 393 and 395, respectively. The wells may be connected by microchannels or isolated without a connection. Alternately, one or more of pins 383 may be positioned into the solution, in a lateral center (see FIG. 12) of the associated well 387, or laterally offset closer to a side wall 397 of the well (see FIG. 10). The heater is removably positioned against the plate or vice versa.

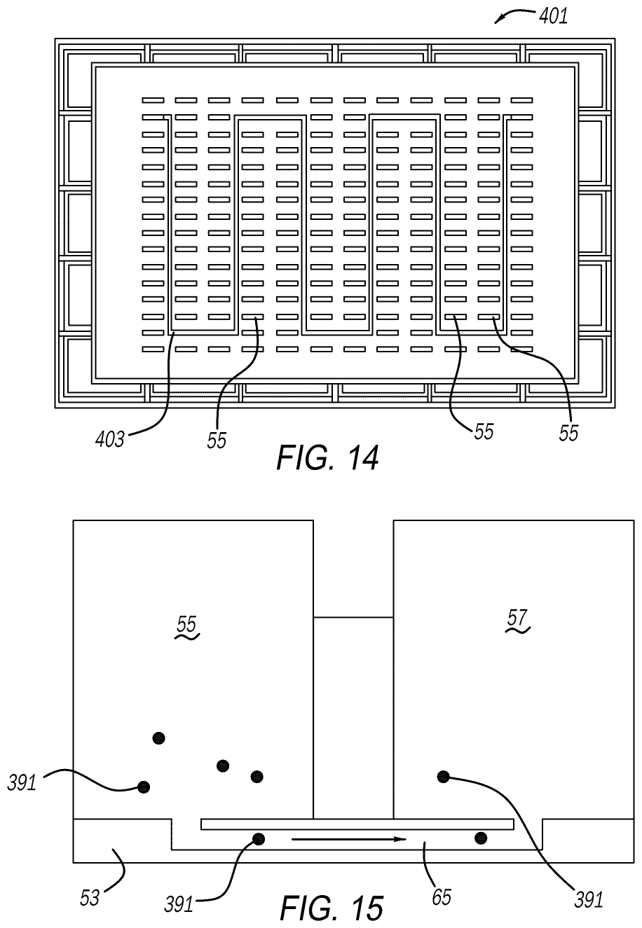

Various serpentine temperature controller embodiments of the present apparatus are depicted in FIGS. 13, 14 and 28-30. FIGS. 13 and 14 show a serpentine temperature control system 401 includes serpentine curved channels 403 cut (e.g., by machining) or formed (e.g., by injection molding or additive 3D printing) in a bottom surface of plate 53, with a pump attached to circulate heated fluid or coolant fluid through the channels. The serpentine channels are also referred to herein as thermal channels or conduits. The cell culture wells are depressed into an opposite upper surface of the plate. In one exemplary configuration, the serpentine channel has generally straight segments, each running parallel between two columns of the wells, perpendicularly joined by generally straight end segments located between rows of the wells. The intersections of the segments may be curved.

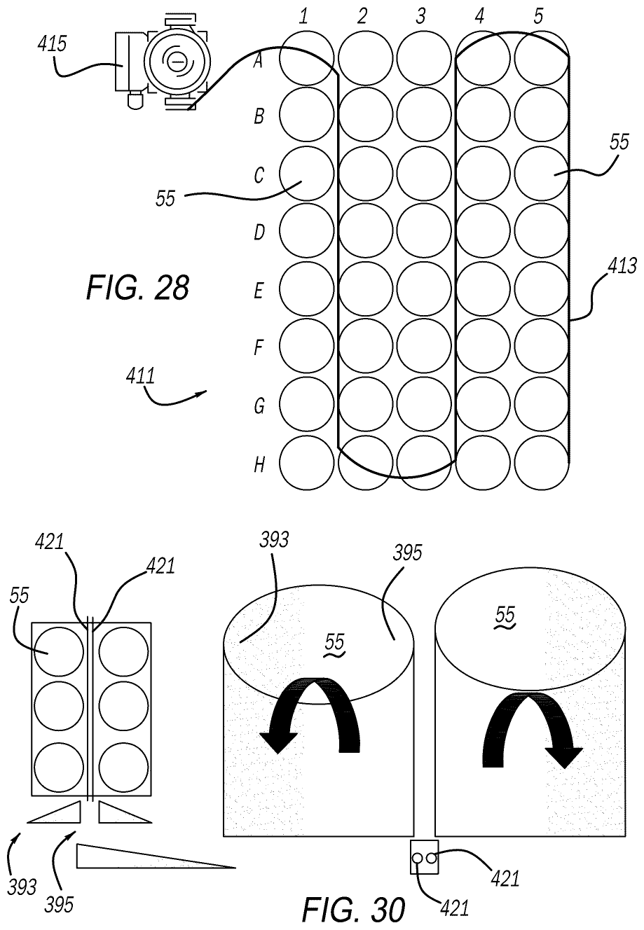

The FIG. 28 variation of the serpentine temperature control system 411 includes serpentine curved tubes 413 forming channels or conduits through which a pump 415 flows heated or coolant fluid. A variation shown in FIG. 30 depicts a serpentine pattern, running up and down columns of wells 55 in a similar pattern to that of FIG. 14 or 28, but for electrically resistive wires 421, also referenced herein as heating conduits for carrying electricity, connected to a power supply via an electrical circuit. Channels 403, tubes 413 or wires 421, serving as temperature control elements, can be mounted below wells 55 or within the upstanding plate walls between the wells.

Furthermore, in the FIG. 13 example, the thermal serpentine channels 403 are preferably located below the upstanding walls 501 separating the adjacent wells 55 and 57, with the optional connecting microchannels 65 between wells being generally coplanar with all or part of the serpentine channels 403. Alternately, the thermal serpentine channels can be directly below and aligned with a portion of the well(s). Ethanol coolant preferably flows through the thermal serpentine channels via the pump and coupled hoses, but water or other fluids may alternately be used.

Alternate embodiments of the present apparatus and method include generating electromagnetic waves, focusing visible light, generating microwaves, and the like, to serve as temperature control elements to heat the liquid in one or more of the wells. In another alternate embodiment, a Peltier heater with upstanding metal fins projecting between the well side walls, may be used. The plate is preferably made from an injection molded polymeric material, which is inexpensive and optionally, disposable. But the plate may alternately be glass with the wells machined or etched therein.

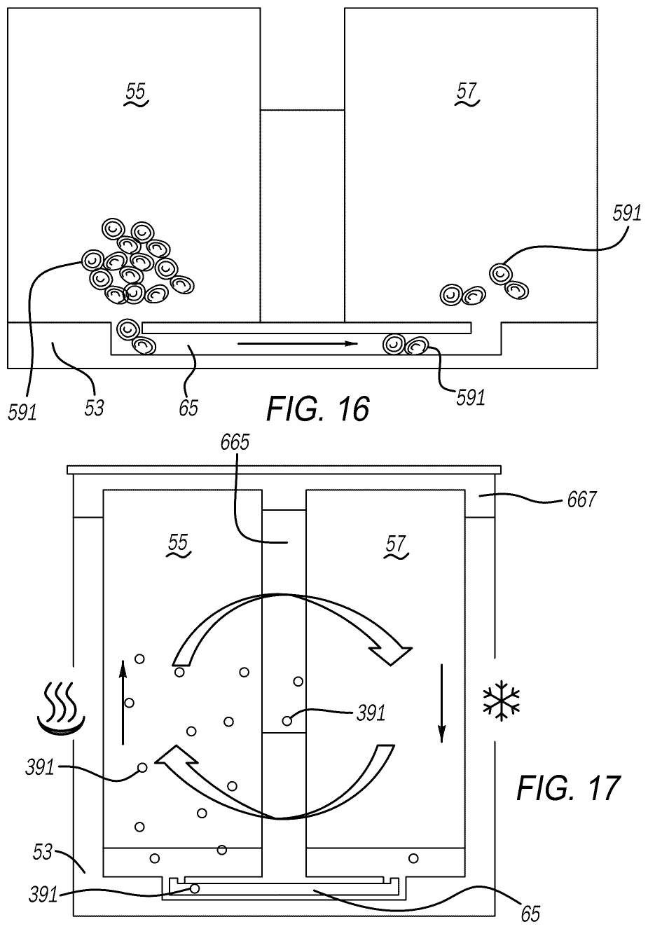

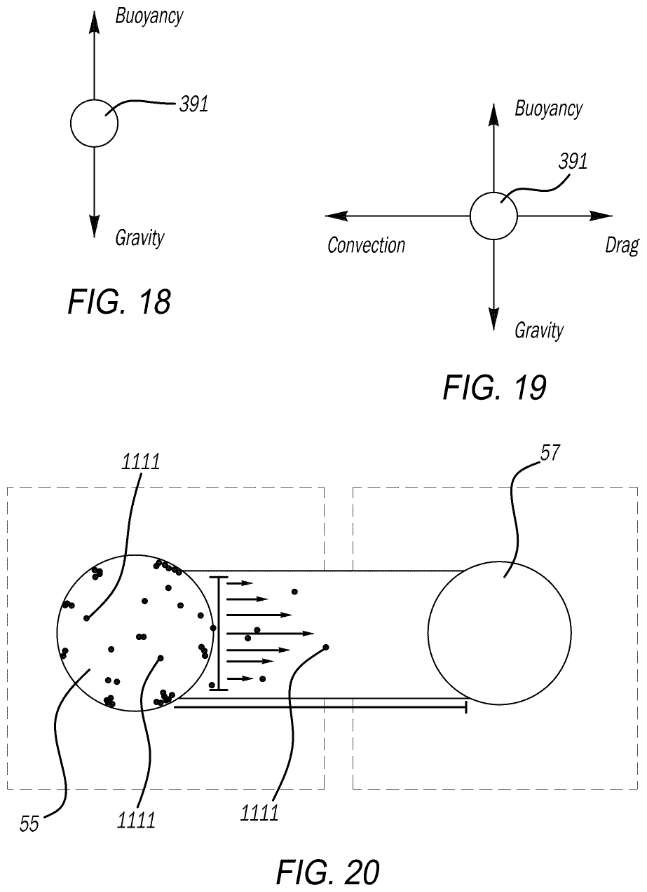

FIG. 15 illustrates convection thermal flow moving cells 391, reagents or the like, from cool well 55 through channel 65 to warm well 57, with the heating and/or cooling being caused by any of the temperature control elements discussed herein. In FIG. 16, the cells are human or animal blood cells 591. Turning now to FIGS. 17-19, the convection thermal flow causes cells 391 within the liquid solution to rotate between cool well 55 and warm well 57 via lower channel 65 and also through an upper channel 665. The top of the wells and upper channel are removably enclosed by a cap 667 attached upon plate 53. Density decreases causing the hot liquid to rise in the warm well, and the density increases causing the cold liquid to sink in the cool well. Each cell 391 reaches a static equilibrium between buoyancy and gravity (FIG. 18) while each cell thermally flows by the convection overcoming drag (FIG. 19). Any of the temperature control elements can be used with this embodiment.

Referring to FIG. 25, a fifth plate embodiment of the present apparatus 751 uses a difference in evaporative cooling rates to create a temperature gradient. This construction includes a cell culture plate 753 with wells 755, 756 and 757 formed therein for holding a cell and liquid solution. Intermediate or middle well 756 is a high evaporation well of a cooler temperature than the outer cell culture wells 755 and 757, thereby causing the closest sides of the outer wells to be cooler than the opposite sides of the other wells. If each well is isolated from each other without any flow-through channel interconnection, then the cells therein swirl between cool zones 793 and warm zones 795 within each well; this is ideally suited for using the present temperature control system with any otherwise conventional plate. A microchannel can be optionally located in the bottom wall of plate, below the wells, and connects between the outer cell culture wells 755 and 757 to cause convective flow of the solution between the connected wells and underneath intermediate well 756.

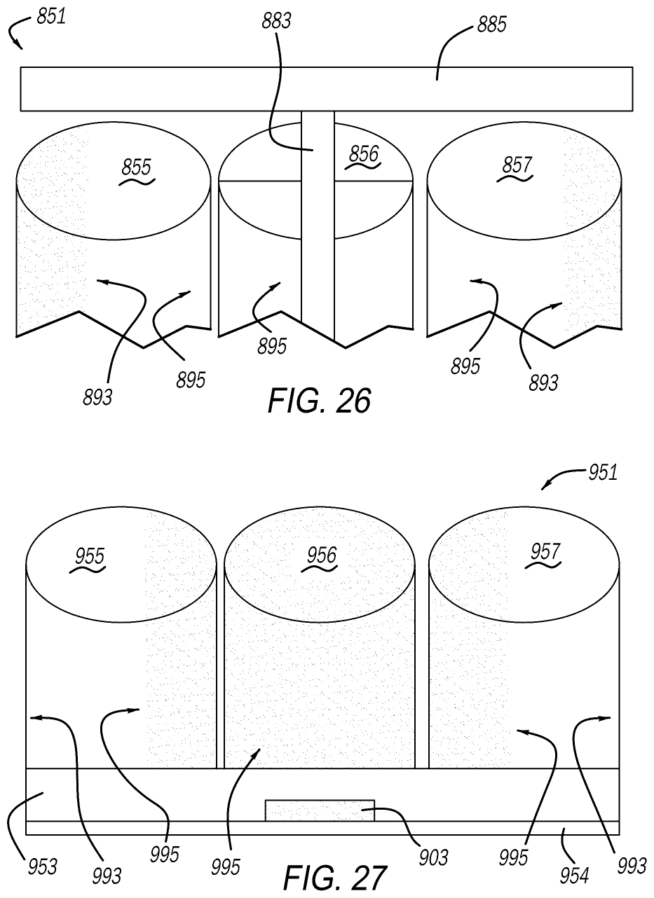

FIG. 26 shows a sixth plate embodiment of the present apparatus 851 uses a heating pin 883, connected to a heating electrical circuit including a generally rigid metallic buss bar 885, as a temperature control element. This construction includes a cell culture plate with wells 855, 856 and 857 formed therein for holding a cell and liquid solution. Pin 883 is inserted within an intermediate or middle well 856 which causes it to be of a hotter temperature than the outer cell culture wells 855 and 857, thereby causing the closest sides of the outer wells to be hotter than the opposite sides of the other wells. If each well is isolated from each other without any flow-through channel interconnection, then the cells therein swirl between cool zones 893 and warm zones 895 within each well; this is ideally suited for using the present temperature control system with any otherwise conventional plate. A microchannel can be optionally located in the bottom wall of plate, below the wells, and connects between the outer cell culture wells 855 and 857 to cause convective flow of the solution between the connected wells and underneath intermediate well 856.

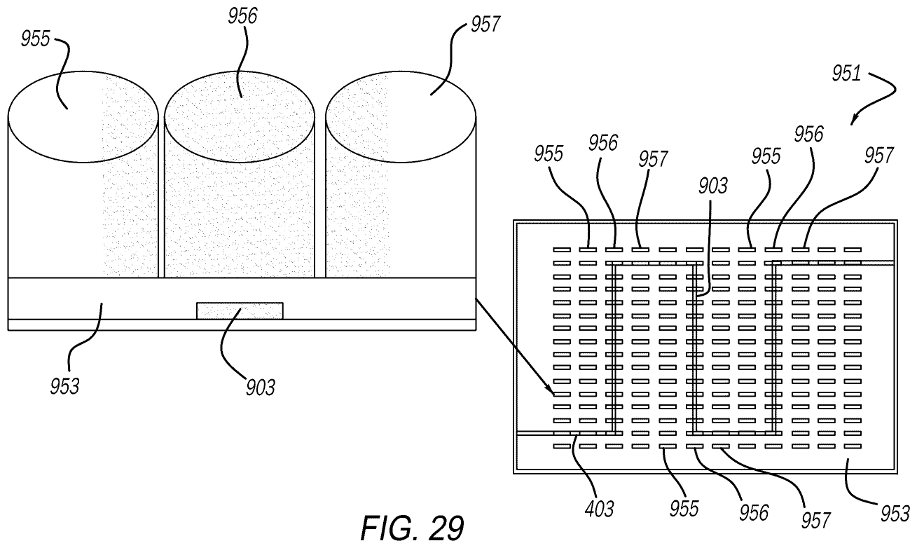

As can be observed in FIGS. 27 and 29, a seventh plate embodiment of the present apparatus 951 uses a coolant channel or conduit 903 molded, machined, 3D printed, etched or otherwise formed within a bottom of a plate 953. A generally flat closure panel 954 is attached to the bottom of plate 953 and seals the channel. A pump circulates coolant fluid through channel 903 below an intermediate well 956, and other wells in the column, so that it serves as a temperature control element. The channel weaves back and forth in a serpentine pattern with primary straight sections aligned with the intermediate wells, being generally parallel to each other. This construction also includes cell culture wells 955 and 957 formed in plate 953 for holding a cell and liquid solution.

Coolant channel 903 causes intermediate well 956 to be of a cooler temperature than the outer cell culture wells 955 and 957, thereby causing the closest sides of the outer wells to be hotter than the opposite sides of the other wells. If each well is isolated from each other without any flow-through channel interconnection, then the cells therein swirl between cool zones 993 and warm zones 995 within each well. A microchannel can be optionally located in the bottom wall of plate, below the wells, and connects between the outer cell culture wells 955 and 957 to cause convective flow of the solution between the connected wells and underneath intermediate well 956. Conversely, the coolant channel may alternately be replaced with a heating channel for heated fluid to flow therein to heat the intermediate well.

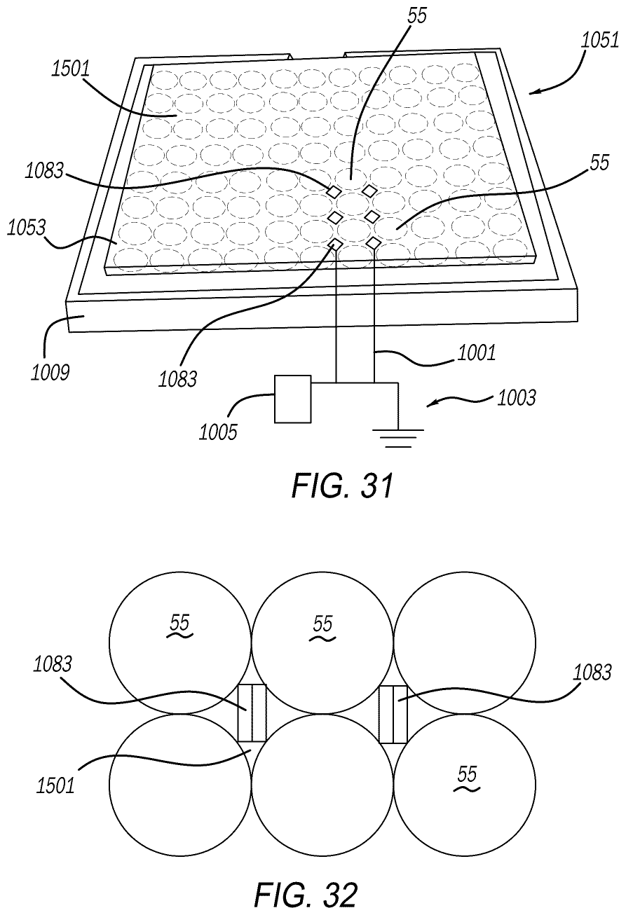

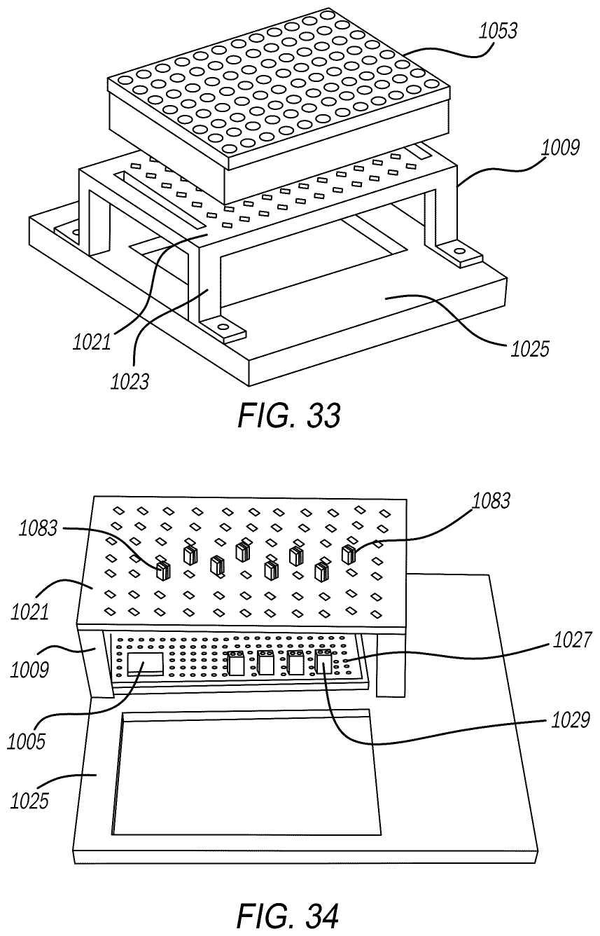

A Peltier temperature control system is employed in an eighth embodiment for the present apparatus 1051 can be seen in FIGS. 31-37. A Peltier electronic component 1083 acts as a heating or cooling element and preferably has a maximum input voltage of about 1.3 Vdc, a maximum input current of about 0.7 A, and an internal resistance of about 1.43 ohms. The Peltier components 1083 are made from a semiconductor material placed between two electrically insulating but thermally conductive plates of ceramic. Each Peltier component 1083 preferably has a peripheral size of 4.3 mm×3.3 mm×2.77 mm, a wide delta-T max, Q-max up to 1.9 W, Au plating; it is suitable for soldering, precise temperature control and of solid-state construction, connected to an electrical circuit 1001 including a power supply 1003 and a programmable or solid-state controller 1005.

Each Peltier component 1083 is located within an upstanding wall 1501 of plate 1053, at an intersection between four adjacent wells 55. A holder or frame 1009, with a raised platform 1021 and upstanding legs 1023 attached to a base 1025, is employed below plate 1053. Controller 1005 is preferably a microprocessor mounted to a printed circuit board 1027 upon which are mounted terminal blocks 1029 for electrically connecting to Peltier components 1083. Optionally, microchannels may interconnect multiple adjacent wells 55.

It is noteworthy that any of the previous embodiments allow for different thermal control in different areas of the well plate or tray. For example, heating of the outermost rows and/or columns of wells via pins, tubes and/or wires a different temperature than heating of inner rows or columns of wells, overcomes edge effect temperatures from the ambient air temperature acting on the periphery of the plate. This different heating and/or cooling row-by-row and/or column-by-column control, via real-time thermal sensors on different plate locations and programmed microprocessor control of the different temperature elements, beneficially allows for broad area-by-area variations or alternately, locally focused area-by-area variations, which may be predetermined or automatically varied during the cell culture processing or test procedure. While evaporation plays a role in traditional temperature edge effects, it is believed that uneven heating/cooling across the plates also leads to differences between the inner and outer wells. Therefore, the present apparatus allow the plate to reach a temperature equilibrium quickly, reducing differences across the plate, and increase reproducibility across the plate.

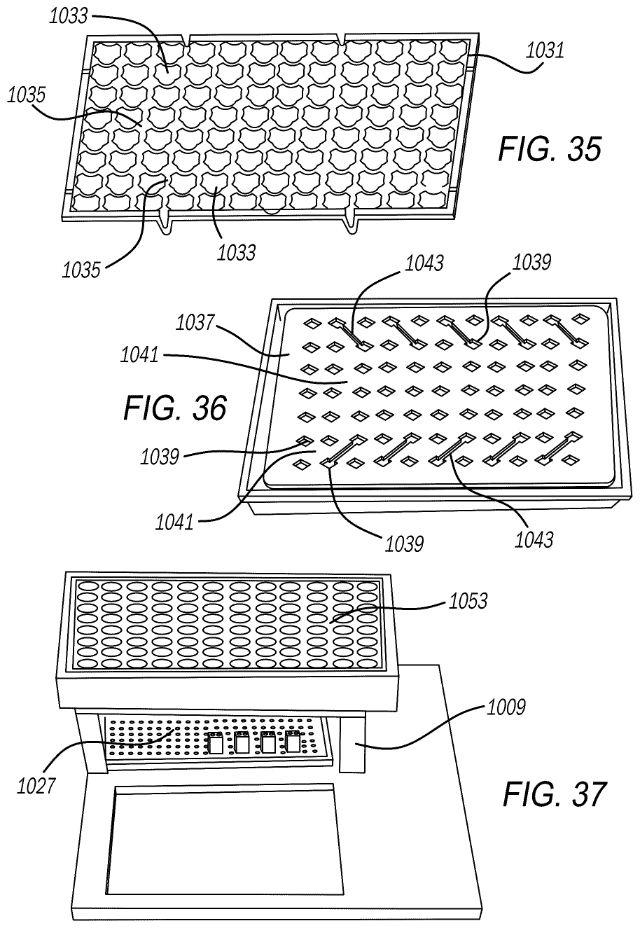

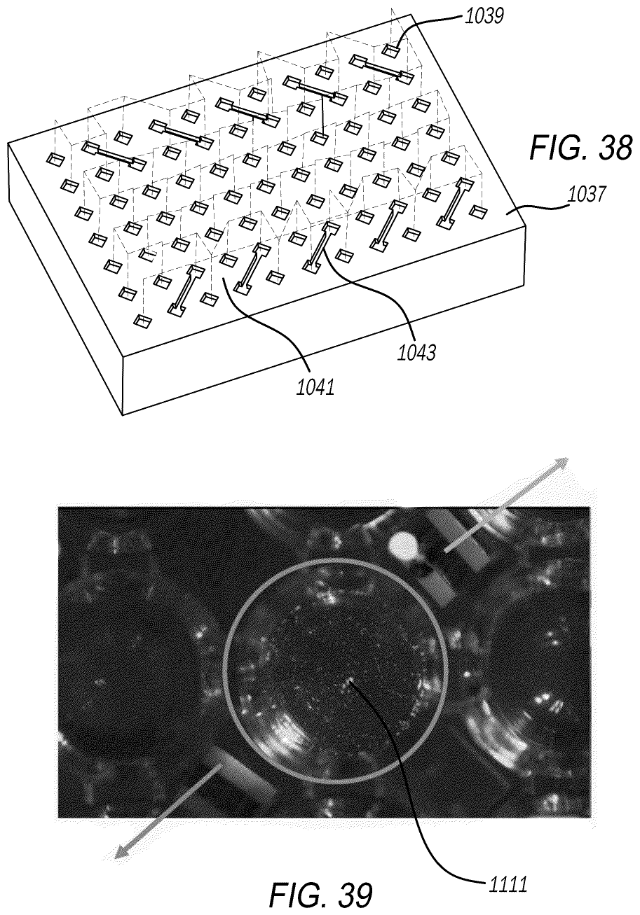

FIG. 35 shows a bottom mold 1031, made of a ceramic material, for forming a bottom or underside of a 96 well plate 1053. Bottom mold 1031 includes recesses 1033 for forming the wells and upstanding walls 1035 therebetween. FIG. 36 shows a ceramic top mold 1037 made on a milling machine with pockets 1039 and upstanding side walls 1041 between the pockets, and elongated and diagonally oriented engraves 1043 between some adjacent pockets. Moreover, FIG. 38 illustrates a tool path for milling pockets 1039 into the ceramic material for the Peltier components to fit into. Also, shown is the detailed milling pattern for elongated rectangular engraves 1043 for further detailed insulation. Then, FIG. 37 shows the 96 well plate 1053, formed between the molds, attached to the frame 1009 and printed circuit board 1027 for the Peltier components.

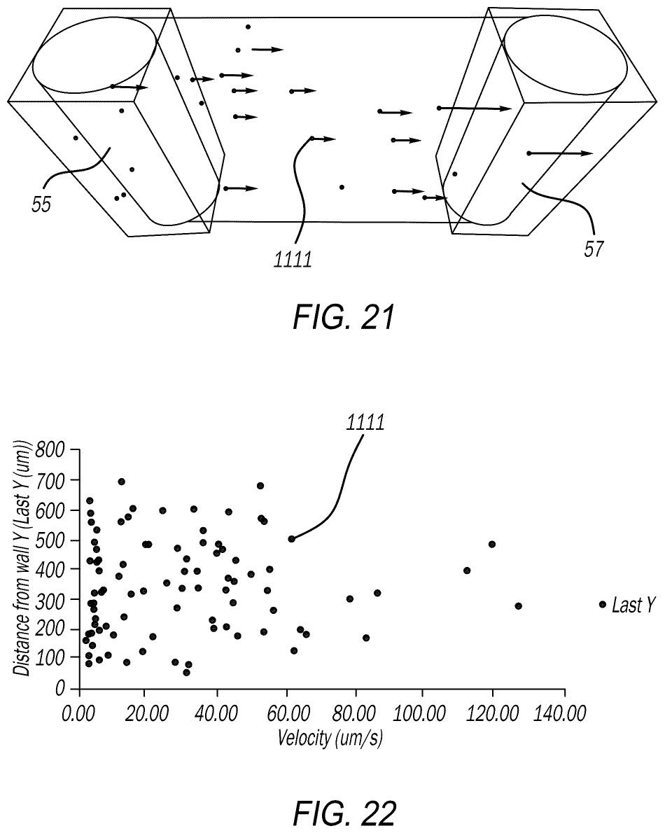

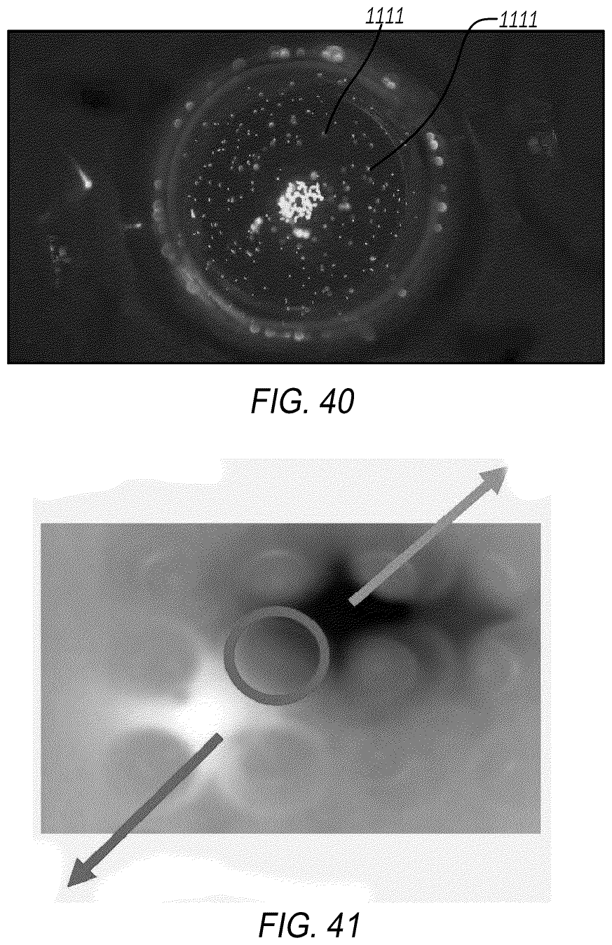

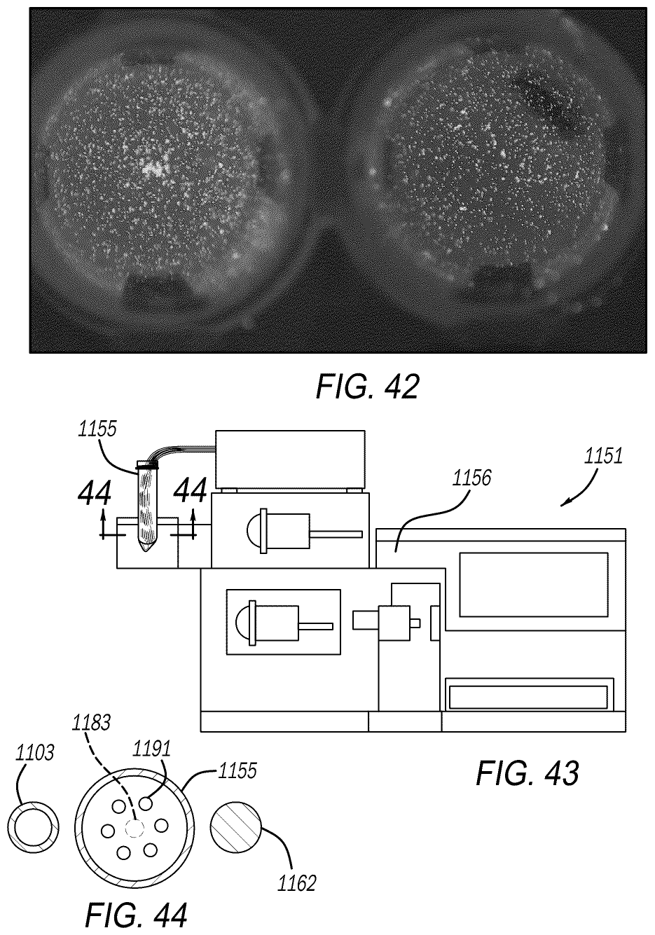

Exemplary expected tests are now discussed with reference to FIGS. 20-24 and 39-42. The preceding first through fourth embodiments generate convection currents within a standard 5 ml reagent reservoir using a thermal gradient. The gradients are generated by a device constructed of 2-part epoxy that was poured and cured into a commercially available cell reservoir. The hardened molded epoxy was then cut in half along the sagittal plane to create two duplicate parts. These parts fit together and allow an unmodified reservoir to fit tightly over them.

Furthermore, thermal gradients are generated by heating or cooling the epoxy components before fitting to the unmodified reservoir. Gradients are observed using mismatched (one hot, one cold) or matching (cold-cold) components with warmer temperate water in the reservoir. Therefore, the thermal gradient is created when some combination of the reservoir and the media show temperature differences. Flow is observed using fluorescent beads (simulating cells) matched to the density of the media and imaged with a fluorescent microscope. This technology can be adapted for other reservoir volumes (e.g., 25 ml, 100 ml) and the geometry of the reservoir could be modified to better facilitate the generation of convection currents and flow patterns. This would lead to a device paired with a custom consumable reservoir. The material used to make the device could be modified to provide more effective heat transfer and increase thermal retention. Multiple methods of heating, cooling, and maintaining temperature in the device are proposed including but not limited to: electric heating and cooling (e.g., Peltier chip), radiator heating, liquid cooling, heat exchangers, insulation, fans, heat cable, heated base.

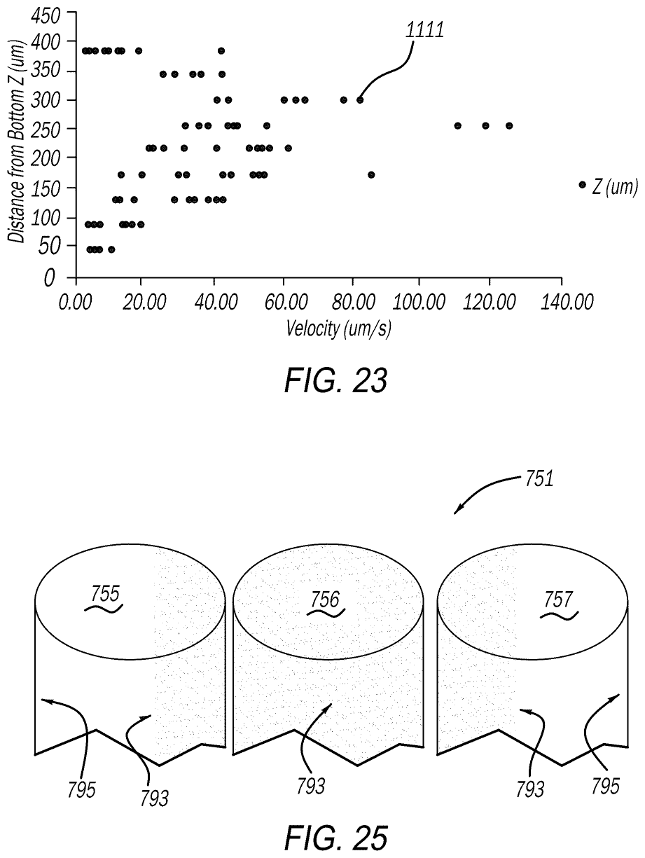

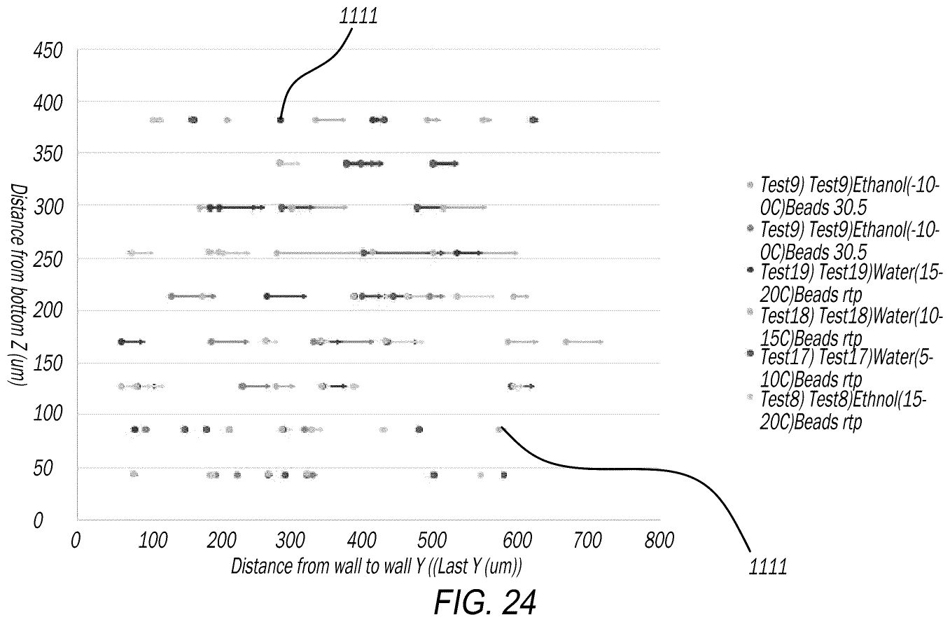

In some exemplary configurations, such as that illustrated in FIGS. 23 and 24, fluorescent beads and media of matched density within the device channels quantify fluid dynamics in real time using confocal fluorescence microscopy and quantified bead velocity using segmentation and image analysis. Velocity vectors align in one direction, with longer vectors at the center and shorter ones at the channel edges indicative of laminar flow. This creates predictable flow that will generate sheer stress on cultured cells, transport secreted factors in a directional manner, stabilized oxygen tension and removal of waste due to continued flow. The expected results reveal a consistent trend where greater changes in X values (movement down the channel) are observed at central Z positions within the total depth of 425 μm. This demonstrates a correlation between the magnitude of velocity, the bead's position within the channel confirming the presence of central laminar flow. FIGS. 39-42 depict fluorescent and thermal imaging of beads in circulation imaged through fluorescent microscope with Peltier heating effects (diagonal bottom left arrow) and Peltier cooling effects (diagonal right arrow) occurring on the well. It is believed that heating a portion of the solution to at least 37° C. (for an isolated well) or heating one of the wells to at least 37° C. (for interconnected wells) is ideally suited for achieving consistent direction flow and greatest cell displacement. Nevertheless, well and/or solution temperatures of at least 15° C. may be satisfactory.

Referring to FIGS. 43 and 44, a ninth embodiment for the present apparatus 1151 uses a generated flow to maintain cells 1183 in suspension within a liquid solution inside a well, more specifically a glass test tube 1155. Test tube 1155 is connected to an automated multimode reagent dispenser 1156, such as a Multiflo FX machine (from Agilent BioTek Inc.). The heating is applied to one side of the conical tube 1155 via any of the heating elements 1162 discussed herein (with or without cooling via any of the cooling elements 1103 discussed herein, on the opposing side), inducing a temperature gradient within the suspension. Alternatively, it could be applied from inside the tube from a heating element 1183 aligned with the central axis of the tube. This could be built into the intake that houses the tubes extending between dispenser machine 1156 and test tube 1155.

Dispenser machine 1156 is an automated liquid handler that employs the present convection thermal swirling to replace a traditional magnetic stir bar inside test tube 1155; this avoids the stir bar increase in void volume and introduction of a foreign component into the media. A variation may involve sampling from other common lab wear (i.e., 0.5 mL PCR tubes, 1.5 mL microcenterfuge tubes, etc.) where a device is created to mix and maintain suspension/mixing of multiple tubes/samples. For example the machine may take cell from a tube, such as an exemplary 50 mL conical plastic centrifuge tube, and dispenses them into wells of a microplate. Alternately, the array of wells within a microplate can heated or cooled as was discussed with many of the embodiments described hereinabove. Alternately, machine 1156 may instead be a flow cytometry machine for biological cells or a biological cell sorting machine, which sample suspensions of cells via wells in FACS tubes or microplate wells, to be analyzed in the machine.

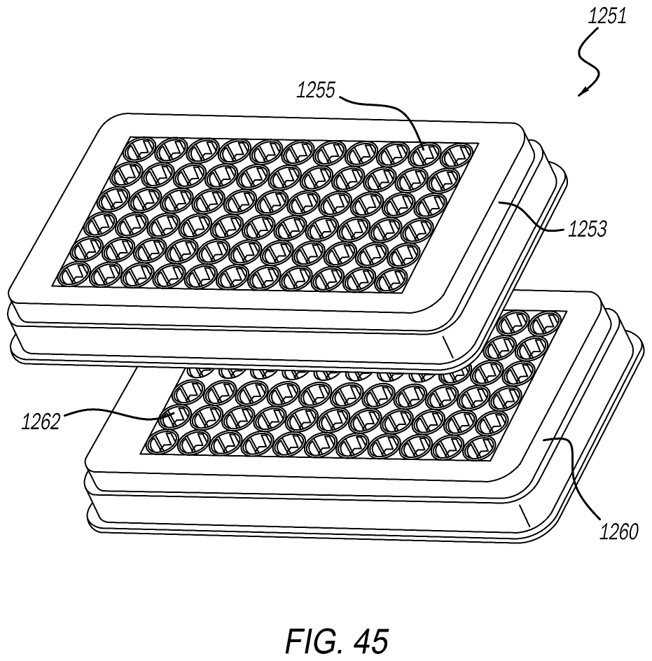

FIG. 45 depicts a tenth embodiment of the present cell retention apparatus 1251. This configuration includes a microplate 1253 having an array of wells 1255 for retaining a biological cell and liquid solution therein. A bottom block or base 1260 is attached to a lower surface of microplate 1253 to maintain an even temperature across wells 1255 in microplate. Furthermore, base 1260 may optionally include cavities 1262 in its upper surface configured to receive an underside of wells 1255 therein in a nested manner. Base 1260 is preferably made from aluminum metal, cast from ceramic, polymeric or other such materials. Any of the previously discussed heating or cooling systems, interconnecting channels, and/or processing machines may be employed with microplate 1253. The base facilitates even heating (or cooling) across the array of wells by the microplate, by insulating it from differential temperature gradients experiences based on well location.

For certain of the preceding embodiments, an exemplary average temperature ranges between 20-37° C. (room temp and cell culture incubator) with an ability to maintain a gradient of <5° C. withing the circulating liquid. The vessel temperature to induce this gradient may be held at significantly higher or lower temperatures. Furthermore, the maximum temperature of the heating devices preferably stays below the glass transition temperatures of plastic vessels they encounter; such as for cyclo-olefin-copolymer <65° C., and for polystyrene <75° C. Also, certain of the pin configurations are preferably made of thermally conductive materials, such as ceramics, metals, and the like. Moreover, in some of the embodiments, the plate is made from polystyrene, cyclo-olefin-copolymer, or cyclo-olifin polymer. Various of the present microplates can be manufactured via over-molding polymeric material over a metal scaffold to integrate thermal transfer materials withing a well plate. Nevertheless, other polymers may be used, such as polypropylene when chemical resistance is important, and a metallic well plate may offer superior heat transfer, thereby potentially benefiting performance.

In conclusion, the present thermal driven flow in wells for cell cultures maintains homogeneity in cell culture media while speeding up chemical reactions therein. It is contactless, which eliminates the need for pumps connected to the well solution or undesired shaking or rocking of the well. Some of the present temperature control elements may be used for conventional cell culture microplates while other employ a uniquely designed plate.

While various embodiments of the present apparatus and method have been disclosed, it should be appreciated that other variations may be made. For example, different well shapes (such as circular, elongated, polygonal) and plate characteristics (such as will upstanding peripheral edges, grooves between wells, different port locations) may be employed, although such alternatives may not realize all of the advantages of the preferred configurations. Also, different electrical circuitry and fluid flow paths may be employed, although some of the benefits may not be realized. The features of any of the embodiments may be mixed and matched in an interchangeable manner with any of the other embodiments disclosed herein, and the dependent claims may be multiply dependent on any of the other dependent claims in any combination. Various changes and modifications are not to be regarded as a departure from the spirit or the scope of the present invention.

The invention claimed is:

1. A cell retention apparatus comprising:
a plate including multiple cell retention wells configured to hold a liquid and biologic cell solution therein;
a channel connecting a set of the wells; and
at least one temperature control element located in or adjacent to the set of the wells being configured to heat or cool a portion of the set of the wells to cause thermal convection flow of the solution in or between the set of the wells via the channel.

2. The apparatus of claim 1, wherein:
the channel is integrally formed within a bottom wall of the plate below the set of the wells;
the plate is a disposable polymeric material;
the plate includes multiple parallel and spaced apart columns and rows of the wells; and
the at least one temperature control element comprises a plurality of heaters, each corresponding to one of the wells.

3. The apparatus of claim 1, wherein the at least one temperature control element includes an elongated and conductive pin extending into the liquid inside a corresponding one of the wells, and the pin is located offset from but substantially parallel to a centerline of the corresponding one of the wells.

4. The apparatus of claim 1, wherein the at least one temperature control element includes an elongated and conductive pin extending into the liquid inside a corresponding one of the wells, and the pin extends at least half but less than an entire depth of the corresponding one of the wells.

5. The apparatus of claim 1, wherein the at least one temperature control element includes an elongated and conductive pin extending into a polymeric side wall between adjacent of the wells, and the pin is external to the wells.

6. The apparatus of claim 1, wherein the at least one temperature control element comprises an elongated tube that extends in a serpentine path adjacent to rows or columns of the wells in a top view, the tube carries a heating or cooling fluid which is different than the liquid in the wells, and the fluid varying a temperature of the liquid in at least some of the wells.

7. The apparatus of claim 1, wherein the at least one temperature control element comprises an elongated wire that extends in a serpentine path adjacent to the wells, and the wire varying a temperature of the liquid in at least some of the wells.

8. The apparatus of claim 1, wherein the at least one temperature control element comprises multiple Peltier components connected to an electrical circuit, each of the Peltier components being coupled within pockets of the plate adjacent to but external to the wells.

9. The apparatus of claim 1, wherein the at least one temperature control element comprises multiple heating elements, at least one of the heating elements is configured to heat a first group of the wells to a different temperature than a second group of the wells by at least a second of the heating elements.

10. The apparatus of claim 1, wherein the channel spans below an intermediate one of the wells which is not connected to the set of wells interconnected by the channel.

11. The apparatus of claim 1, wherein the temperature control element includes a desiccant located in at least one of the wells.

12. The apparatus of claim 1, further comprising a base attached to a bottom of the plate, the base including cavities in an upper surface thereof within which an underside surface of the wells nest.

13. A cell retention apparatus comprising:
a plate including multiple cell retention wells configured to hold a liquid and biologic cells therein;
a heater or cooler element located between the wells being configured to heat or cool a portion of the wells adjacent to the element to cause thermal convection flow of the liquid and the cells in the wells.

14. The apparatus of claim 13, wherein the element includes an elongated and conductive pin extending into the liquid inside a corresponding one of the wells, and the pin is located offset from but substantially parallel to a centerline of the corresponding one of the wells.

15. The apparatus of claim 13, wherein the element includes an elongated and conductive pin extending into the liquid inside a corresponding one of the wells, and the pin extends at least half but less than an entire depth of the corresponding one of the wells.

16. The apparatus of claim 13, wherein the element includes an elongated and conductive pin extending into a polymeric side wall between adjacent of the wells, and the pin is external to the wells.

17. The apparatus of claim 13, wherein the element comprises an elongated tube that extends in a serpentine path adjacent to rows or columns of the wells in a top view, the tube carries a heating or cooling fluid which is different than the liquid in the wells, and the fluid varying a temperature of the liquid in at least some of the wells.

18. The apparatus of claim 13, wherein the element comprises an elongated wire that extends in a serpentine path adjacent to the wells, and the wire varying a temperature of the liquid in at least some of the wells.

19. The apparatus of claim 13, wherein the element comprises multiple Peltier components, each of the Peltier components being located within a pocket of the plate adjacent to but external to the wells, and being connected to a common power supply and a programmable controller by an electrical circuit.

20. The apparatus of claim 13, wherein the element comprises multiple heating elements attached to the plate, at least one of the heating elements is configured to heat a first group of the wells to a different temperature than a second group of the wells by at least a second of the heating elements.

21. A cell retention apparatus comprising:
a plate including multiple cell retention wells;
a first heater located in or adjacent a first group of the wells;
a second heater located in or adjacent to a second group of the wells;
the heaters being configured to heat a portion of each of the wells; and
the first heater being configured to heat the first group of the wells to a different temperature than that of the second heater with regard to the second group of the wells.

22. The apparatus of claim 21, further comprising:
a channel interconnecting a set of the wells, the channel being integrally formed within a base wall of the plate and below the set of the wells;
the plate being metallic or ceramic; and
at least one of the heaters causing convective flow of a biologic cell and liquid solution between the pair of the wells via the channel.

23. The apparatus of claim 21, further comprising a programmable controller and thermal sensors connected to the heaters, the thermal sensors being attached to the plate, wherein the first group of the wells is closer to a peripheral edge of the plate than is the second group of the wells.

24. The apparatus of claim 21, wherein at least one of the heaters includes an elongated and conductive wire encapsulated within a wall of the plate adjacent to at least some of the wells.

25. The apparatus of claim 21, wherein at least one of the heaters includes an elongated metallic pin extending into cell and liquid solution retained inside a corresponding one of the wells.

26. The apparatus of claim 21, wherein at least one of the heaters includes an elongated metallic pin extending into a side wall between adjacent of the wells but external to the wells.

27. The apparatus of claim 21, wherein at least one of the heaters comprises a conduit that extends in a serpentine path between or below rows or columns of the wells.

28. A cell retention apparatus comprising:
a plate including multiple wells configured to hold biologic cells in a liquid therein;
a temperature control conduit that extends in a serpentine path between or below rows or columns of the wells; and
the conduit heating or cooling a portion of each of the wells adjacent to the to the conduit to cause thermal convection flow of the liquid in the wells.

29. The apparatus of claim 28, wherein the conduit comprises at least five parallel and spaced apart, straight tubular sections with at least two of the columns of the wells being located between each adjacent pair of the straight tubular sections, and a cooling or heating fluid being pumped through the conduit.

30. The apparatus of claim 28, wherein the conduit including an electrically conductive wire attached to the plate and being configured to heat the adjacent of the wells.

31. The apparatus of claim 28, wherein the conduit is located internal to the plate but separate from the wells with a portion of the plate located between the conduit and the wells.

32. A cell retention apparatus comprising:
a plate including multiple retention wells configured to hold a biologic cell and liquid solution therein; and
a base attached to a bottom of the plate, the base including cavities in an upper surface thereof within which an underside surface of the wells nest;
the base being configured to facilitate even heating or cooling across the wells by insulating the wells from differential temperature gradients otherwise experienced based on well location.

33. The apparatus of claim 32, further comprising a heater or cooler being configured to heat the solution in at least some of the wells.

34. A cell retention apparatus comprising:
a cell processing machine;
a cell retention well connected to the machine; and
a heater located adjacent to the well causing convective flow of a liquid within the well when the heater is energized.

35. The apparatus of claim 34, wherein the machine is an automated multimode reagent dispenser, the well is within a test tube or a plate containing an array of the wells, and the well retains biologic cells in a liquid.
